(19) United States
(12) Patent Application Publication
(10) Pub. No.: US 2026/0162818 A1
(43) Pub. Date: Jun. 11, 2026

(54) WEARABLE DEVICES WITH ENVIRONMENT-SPECIFIC BIOMETRIC DETECTION

(71) Applicant: Scott A. Elrod, Lake Jackson, TX (US)

(72) Inventor: Scott A. Elrod, Lake Jackson, TX (US)

(21) Appl. No.: 18/975,955

(22) Filed: Dec. 10, 2024

(51) Int. Cl. G16H 40/67; G16H 50/30

(52) U.S. Cl. CPC G16H 40/67; G16H 50/30

(57) ABSTRACT

A wearable device can include a housing defining a display aperture and an interior volume, the housing including a bottom-face; a display positioned within the display aperture; a pulse oximeter positioned at least partially within the interior volume and oriented opposite the display aperture to send and receive optical signals through the bottom face; a processor disposed within the interior volume and communicatively coupled to the pulse oximeter; and a memory device storing computer-executable instructions that, when executed by the processor, cause the processor to receive optical data from the pulse oximeter; identify an altitude-adjusted oxygen saturation factor specific to an altitude of the wearable device; determine an altitude-adjusted oxygen saturation level based on the optical data and the altitude-adjusted oxygen saturation factor; and provide, at the display, a graphical user interface including an oxygen indicator representing the altitude-adjusted oxygen saturation level.

### FIELD

The described embodiments relate generally to a wearable device, system, and method for biometric detection specific to an environment.

### BACKGROUND

Advancements in wearable device technology continues to proliferate and aid people in a wide array of applications. Many people use wearable devices, for example, to further their health and fitness goals, sports training, body awareness, personal safety, emergency preparedness, communication connectedness, and hobby integrations. Certain wearable devices can identify biometric markers of individuals, as performed by some activity trackers and smartwatches, for instance. Unfortunately, however, the biometric data collected and displayed by such devices can be entirely independent of (or unrelated to) a user's physical environment. Thus, biometric data of conventional wearables can be considered a type of “dumb” or agnostic data that does not account for the specific environment of the user—thereby lending to inaccuracies, false applications, and overall poor translation to user understanding of their own biometric data. Accordingly, there is need for technical improvements to wearable devices that allow environment-specific biometric detection (heretofore unachieved).

The subject matter claimed herein is not limited to embodiments that solve any disadvantages or that operate only in environments such as those described above. Rather, this background is only provided to illustrate one example technology area where some embodiments described herein may be practiced.

### SUMMARY

An aspect of the present disclosure relates to a method that includes determining a real-time availability of atmospheric oxygen for a location based on environmental conditions; identifying measured biometric data of a user currently positioned at the location or within a threshold proximity of the location; determining an environment-adjusted biometric threshold specific to the location; and generating, for display in a graphical user interface of a wearable device, a visualization based on the measured biometric data and the environment-adjusted biometric threshold.

In some examples, the visualization includes a recommendation regarding user exertion. In particular examples, the visualization includes a recommendation to increase oxygen intake. In at least some examples, the visualization includes an indicator graphically representing the biometric data in relation to the environment-adjusted biometric threshold. In one or more examples, the indicator includes an altitude-adjusted vital sign, and the visualization further includes an altitude indicator for the location. In certain implementations, the measured biometric data includes an oxygen saturation (SpO2) value, and the environment-adjusted biometric threshold includes at least one of a threshold SpO2 value or a range of SpO2 values indicative of a user status specific to the location. In one example, the user status includes at least one of normal oxygen levels detected, low oxygen levels detected, hypoxemia detected, or medical attention required.

In some examples, the measured biometric data is a first type of biometric marker, and the method further includes identifying additional measured biometric data of a second type of biometric marker different than the first type of biometric marker; and determining, for the additional measured biometric data, an additional environment-adjusted biometric threshold specific to the location. In certain examples, the method can include receiving travel data including at least one of a destination location or a travel route; estimating a change in potential availability of atmospheric oxygen at the destination location or along the travel route; and generating a predicted health status at the destination location or at a forthcoming location along the travel route based on the measured biometric data and the change in potential availability of atmospheric oxygen. In one or more examples, the environmental conditions include altitude above sea level, and the method further includes using global positioning system (GPS) coordinates to determine the location; identifying the altitude above sea level based on the location; and determining the real-time availability of oxygen as an amount of oxygen concentration at the altitude above sea level. In particular examples, the environmental conditions include at least one of altitude, oxygen concentration, or barometric pressure; and the method further includes using a sensor to analyze the environmental conditions, the sensor including at least one of an oxygen sensor, an altimeter, or a pressure sensor.

Another aspect of the present disclosure relates to a system. The system can include a wearable device and a computing device communicatively coupled to the wearable device. The computing device can include a processor and a memory device storing computer-executable instructions that, when executed by the processor, cause the processor to retrieve, for a real-time location of the computing device, environmental conditions via a network connection to a network device; determine a real-time availability of atmospheric oxygen for the real-time location based on the environmental conditions; determine an environment-adjusted vital sign factor specific to the location; and transmit the environment-adjusted vital sign factor to the wearable device.

In some examples, the memory device includes computer-executable instructions that, when executed by the processor, cause the processor to receive, from the wearable device, environment-adjusted vital sign data of a user, the environment-adjusted vital sign data including vital sign data of the user that is modified according to the environment-adjusted vital sign factor. In particular examples, the memory device includes computer-executable instructions that, when executed by the processor, cause the processor to generate a user recommendation based on the environment-adjusted vital sign data. In certain examples, the user recommendation is based on a travel route to a destination location.

Yet another aspect of the present disclosure relates to a wearable device. The wearable device can include a housing defining a display aperture and an interior volume, the housing including a bottom-face; a display positioned within the display aperture; a pulse oximeter positioned at least partially within the interior volume and oriented opposite the display aperture to send and receive optical signals through the bottom face; a processor disposed within the interior volume and communicatively coupled to the pulse oximeter; and a memory device storing computer-executable instructions that, when executed by the processor, cause the processor to perform certain acts. In some examples, the acts can include receiving optical data from the pulse oximeter; identifying an altitude-adjusted oxygen saturation factor specific to an altitude of the wearable device; determining an altitude-adjusted oxygen saturation level based on the optical data and the altitude-adjusted oxygen saturation factor; and providing, at the display, a graphical user interface including an oxygen indicator representing the altitude-adjusted oxygen saturation level.

In some examples, the wearable device includes a haptic feedback actuator, and the wearable device stores computer-executable instructions that, when executed by the processor, cause the processor to transmit a signal to the haptic feedback actuator in response to determining the altitude-adjusted oxygen saturation level satisfies a predetermined altitude-adjusted oxygen saturation level, the signal configured to actuate the haptic feedback actuator. In some examples, the wearable device includes a speaker, and the wearable device stores computer-executable instructions that, when executed by the processor, cause the processor to transmit a signal to the speaker in response to determining the altitude-adjusted oxygen saturation level satisfies a predetermined altitude-adjusted oxygen saturation level, the signal configured to induce an audible communication from the speaker. In yet another example, the wearable device includes at least one of a cellular network connection or a satellite network connection, and the wearable device stores computer-executable instructions that, when executed by the processor, cause the processor to transmit a digital communication over the cellular network connection or the satellite network connection in response to determining the altitude-adjusted oxygen saturation level satisfies a predetermined altitude-adjusted oxygen saturation level.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 illustrates a system environment for environment-specific biometric detection in accordance with one or more examples of the present disclosure;

FIG. 2 illustrates a side cross-sectional view of an example wearable device;

FIGS. 3A-3B illustrate a flowchart of a series of acts for performing environment-specific biometric detection in accordance with one or more embodiments of the present disclosure;

FIG. 4 illustrates an example graphical user interface of a wearable device;

FIG. 5 illustrates another example graphical user interface of a wearable device;

FIG. 6 illustrates yet another example graphical user interface of a wearable device; and

FIG. 7 illustrates still another example graphical user interface of a wearable device.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following disclosure relates to a wearable device that alone (or in combination with a computing device and/or a third-party server) can generate biometric related information that is specific to a user's environment. The structural components and/or methods disclosed herein enable the generation of highly accurate and intuitive biometric information for users that can help users better understand how their body is performing in real-time (and on-the-fly) in a specific environment. While conventional devices may display biometric data, such information is agnostic to the environment that the user is located in. By contrast, the disclosed embodiments account for the user's environment and correspondingly generate modified biometric thresholds (and/or modify the biometric data itself) to reflect the user's environment. Under this never-before-achieved approach, the environment's imposed limitations (e.g., decreased atmospheric oxygen availability) can be accounted for in the environment-adjusted biometric information. In turn, a user can quickly identify their own body's unique performance in that specific environment. Thus, because the effects of the user's environment are built into the user-facing display elements, the user experiences no uncertainty as to the effects of the user's environment upon their biometric data—which is not the case for conventional devices where users are either mislead or left to wonder whether their biometric data is normal or skewed because of the environment.

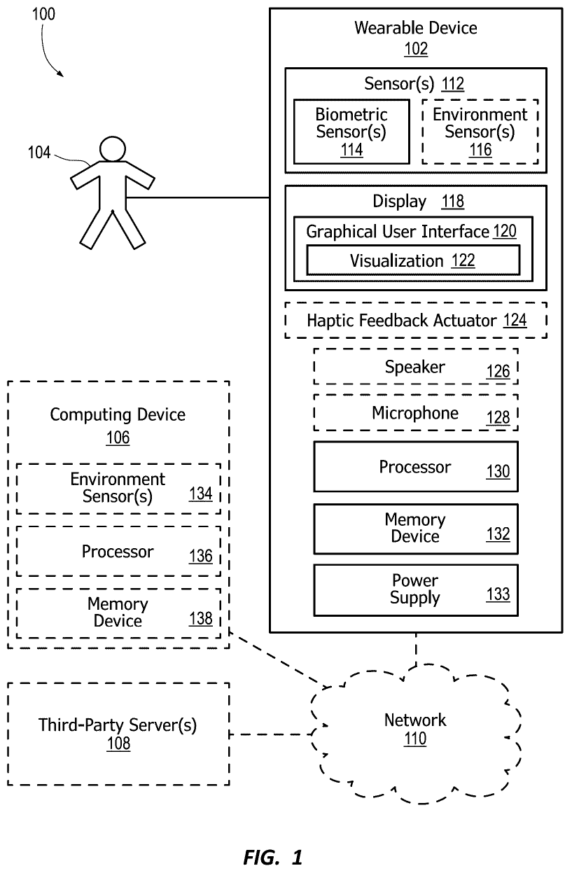

These and other embodiments are discussed below with reference to FIGS. 1-7. However, those skilled in the art will readily appreciate that the detailed description given herein with respect to these figures is for explanatory purposes only and should not be construed as limiting. Furthermore, as used herein, a system, a method, an article, a component, a feature, or a sub-feature including at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1 illustrates a system environment 100 in accordance with one or more examples of the present disclosure. As shown, the system environment 100 can include a wearable device 102 worn by a user 104. Optionally, the system environment 100 can include other system components, including at least one of a computing device 106 or third-party server(s) 108. These components can be communicatively coupled to the wearable device 102 via a network 110. Each is discussed in turn below.

The wearable device 102 can include a wearable electronic device or a portable electronic device. For example, wearable device 102 can be a watch, such as a smartwatch (e.g., a watch having communication capabilities, messaging capabilities, internet capabilities, sensor capabilities, integration with software apps on other devices, etc.) or an electronic watch. The wearable device 102 can include wearable sensors (e.g., heart rate sensors worn on the chest of a user), biosensors, etc. The wearable device 102 may also include wearable medical devices or emergency alert devices (e.g., pendants, bracelets, activity trackers, blood pressure monitors, wearable defibrillators, oxygen supplies, etc.).

In another example, the wearable device 102 can include a head-mountable device. Examples of head-mountable devices can include virtual reality or augmented reality devices that include an optical component (e.g., smart glasses or headsets). In the case of augmented reality devices, optical eyeglasses/glasses or frames can be worn on the head of a user such that optical windows, which can include transparent windows, lenses, or displays, can be positioned in front of the user's eyes. In another example, a virtual reality device can be worn on the head of a user such that a display screen is positioned in front of the user's eyes. The viewing frame can include a housing (e.g., a display housing or display frame) or other structural components supporting the optical components, for example lenses or display windows, or various electronic components.

In yet another example, the wearable device 102 can include wearable acoustic devices (e.g., earbuds, earpieces, hearing aids, earphones, headphones, headsets, ear plugs, noise cancellation devices, etc. that can provide acoustic output and/or ear protection to a user). In some examples, the wearable device 102 can include an earbud having a housing or other portion that can be at least partially disposed in, on, or otherwise in contact with a user's ear (or the area around a user's ear for bone-conduction). The earbud can include one or more electronic components disposed on or within the housing to operate the earbud. These components can include any components used by the earbud to produce audio (or in the case of bone conduction, sound waves or vibrations). For example, electronic components can include one or more speakers, audio drivers, transducers, microphones, processors, power supplies (e.g., batteries), circuitry components including wires, circuit boards, or any other electronic component used in the earbud to generate and output audio.

In a further example, the wearable device 102 can include a human-integrated device that biomechanically connects with human anatomy (e.g., human tissue, bodily systems, organs, nerves, muscles, limbs, etc.). A human-integrated device can include, for instance, a brain-computer interface (e.g., in-vivo implementations that interface with a neural system of a user). A human-integrated device may also include, for example, cochlear implants, bionic eyes, prosthetic limbs, etc.

The wearable device 102 can include a variety of components. The components shown in FIG. 1 are exemplary in nature, and the wearable device 102 is not limited to or required to have these components. One or more of the illustrated components can be omitted, and/or other components can be added to the wearable device 102. Although some components are illustrated as optional via dashed lines, these identifications may also be altered or omitted according to certain applications. For instance, a wearable device comprising earbuds may not include a display, but a wearable device comprising a watch may include a display. Thus, the components of the wearable device 102 may be adapted for the desired application.

In some examples, the wearable device 102 can include one or more sensor(s) 112. The term “sensor” as used herein can refer to one or more sensing devices to gather user data via biometric sensor(s) 114 and/or environment data via environment sensor(s) 116. The biometric sensor(s) 114 can include a variety of sensing devices, such as a camera or imaging device, temperature device, oxygen device, movement device, brain activity device, sweat gland activity device, breathing activity device, muscle contraction device, etc. Some particular examples of sensors include an electrooculography sensor, electrocardiography sensor, EKG sensor, heart rate variability sensor, pulse sensor, blood volume pulse sensor, SpO2 (or blood-oxygen) sensor, pulse oximeter, compact pressure sensor, electromyography sensor, core-body temperature sensor, galvanic skin sensor, etc. Many other sensors are also herein contemplated. For example, the biometric sensor(s) 114 can include glucose monitoring sensors (CGM) or glucometers, thermometers, etc. In these or other examples, the wearable device 102 can include one or more biometric sensor(s) 114. For instance, in certain implementations, the wearable device 102 can include an SpO2 sensor and a pulse sensor.

The wearable device 102 can, in some examples, include one or more of a variety of environment sensor(s) 116 to identify environmental conditions at or near the wearable device 102 (i.e., local conditions). The environment sensor(s) 116 can include, for instance, an accelerometer, gyroscope, magnetometer, inclinometer, pressure sensor, barometer, infrared sensor, global positioning system sensor, gas sensor, oxygen sensor, humidity sensor, temperature sensor, inertial measurement unit, proximity sensor, light sensor, chemical sensor, etc. The local conditions can include wind speed and direction, temperature, pressure, elevation, humidity, precipitation, sunrise/sunset times, weather forecast, latitude and longitude, altitude, oxygen levels, atmospheric compositions, etc. The local conditions can correspond to sensor data obtained via the environment sensor(s) 116 onboard the wearable device 102 and/or from values obtained via the computing device 106 and/or the third-party server(s) 108.

In some examples, the wearable device 102 can include a display 118. The display 118 can include an electronic display or digital display for presenting a graphical user interface 120 and a visualization 122 associated therewith. The display 118, for example, can include a light element as part of a light emitting diode (LED) display, quantum LED (QLED) display, organic LED (OLED) display, liquid crystal display, digital light processing display, plasma panel display, rear-projection display, micro display, touch display, etc. Other examples of the display 118 can include e-ink displays. In some examples, the display 118 is resistant to and operable in various environmental conditions, including rain, snow, extreme temperatures (e.g., below freezing and above 100+ degree Fahrenheit), dust, debris, etc. In yet another example, the display 118 is scratch and/or impact resistant to help mitigate undesired screen damage.

In these or other examples, the display 118 can include the graphical user interface 120 with compatibility to software application programming. The graphical user interface 120, for instance, can provide the user 104 the capability to intuitively operate the wearable device 102 and/or communicate with an external device (e.g., the computing device 106 and/or the third-party server(s) 108, etc.) through manipulation of the display 118 and/or elements coupled to the wearable device 102. For instance, the graphical user interface 120 can include user interfaces for the user 104 to engage with the display 118 (e.g., tap, touch, hold, scroll, slide, pinch, etc.) to perform certain user interface operations.

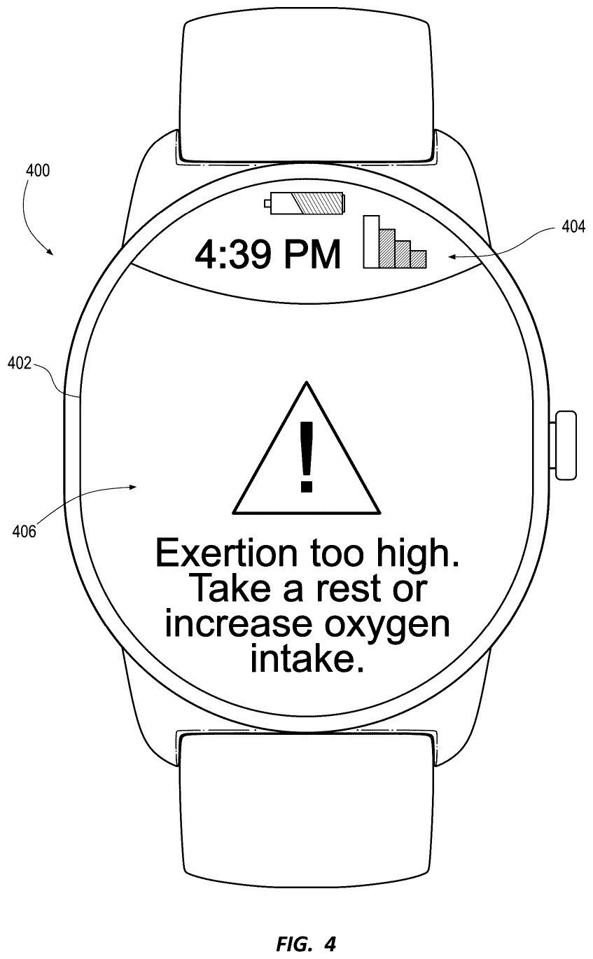

In particular examples, the graphical user interface 120 can include the visualization 122. The visualization 122 can include one or more graphical visualizations, digital representations, interactive elements, or display elements viewable to the user 104 through the display 118. A few examples of the visualization 122 are shown and described in detail below in relation to FIGS. 4-7. In general, however, the visualization 122 can include any data representation that may be relevant or helpful to the user 104. For example, the visualization 122 can include health data, weather data, device status data (e.g., of a scent control apparatus), activity data, hunting data, map or direction data, traffic data, etc. The visualization data 122 can also serve as a secondary display of certain connected devices to display related information (e.g., environmental conditions, scope/turret settings, range finder data, binocular data, etc.). In specific implementations, the visualization 122 can include biometric data, particularly environment-adjusted biometric data (e.g., altitude-adjusted vital signs), as will be explained below. The visualization 122 may be presented in various ways (e.g., via icons, images, symbols, messages, notifications, indicators, charts, metrics, alphanumeric characters, prompts, pop-up windows, and the like.

In addition to, or alternatively to the display 118, the wearable device 102 can include various other components for communicating to the user 104 via other (i.e., non-visual) ways. For example, the wearable device 102 can include a haptic feedback actuator 124. The haptic feedback actuator 124 can include, for instance, an eccentric rotating mass, linear resonant actuator, voice coil actuator, piezoelectric actuator, a motor-assist actuator, etc. In these or other examples, the haptic feedback actuator 124 can provide a vibration, tactile feedback, or the perception of a specific interaction (e.g., the perceived feel of a click-depression, scroll, or swipe). In certain examples, the haptic feedback actuator 124 can coordinate haptic feedback with the visualization 122 (e.g., a vibration generated by the haptic feedback actuator 124 in combination with the visualization 122 generated by the display 118).

In some examples, the wearable device 102 can include at least one of a speaker 126 (e.g., for audio output, sound generation, audible warning signals, text-to-speech generation, etc.) and/or a microphone 128 (e.g., for receiving audio input, voice commands, user dictation, audio/video communications, etc.). In some examples, audio output from the speaker 126 can accompany (or substitute) the presentation of the visualization 122 at the display 118. For example, the audio output may include an audible warning chime that coincides with the visualization 122. Similarly, the microphone 128 can convert audio signals (e.g., a user dictation or sound recording) to a digital emergency message for transmitting to an external device, such as the third-party server(s) 108 in response to the generation of the visualization 122 for presentation at the display 118.

In one or more examples, the wearable device 102 can include a processor 130. The processor 130 can include a system on chip, integrated circuit, driver, microcontroller, application processor, crossover processor, etc. The processor 130 can also include circuitry and associated circuit boards, connectors that electrically couple components together, or other suitable electronic components (e.g., resistors, capacitors, inductors, potentiometers, transformers, diodes, transistors, etc.). In these or other examples, the processor can execute computer-executable instructions received from the memory device 132, another component of the wearable device 102, the computing device 106, and/or the third-party server(s) 108. Additionally or alternatively, the processor 130 can receive a signal from one or more components (e.g., the sensor(s) 112).

In response to a received signal and/or executing the computer-executable instructions, the processor 130 can transmit a signal to one or more components. For example, the processor 130 can transmit a signal to the display 118 to generate (or update) the visualization 122. In another example, the processor 130 can transmit a signal to the haptic feedback actuator 124 to generate a haptic response, a signal to the speaker 126 to generate an audio response, and/or a signal to the microphone 128 to process sound signals.

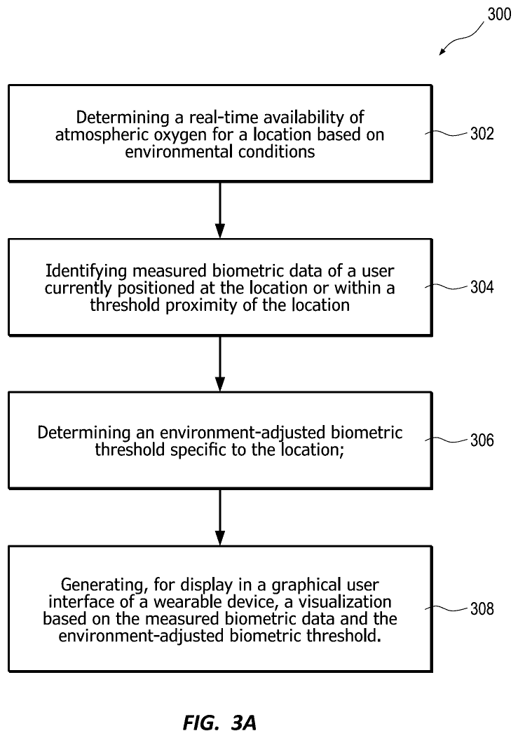

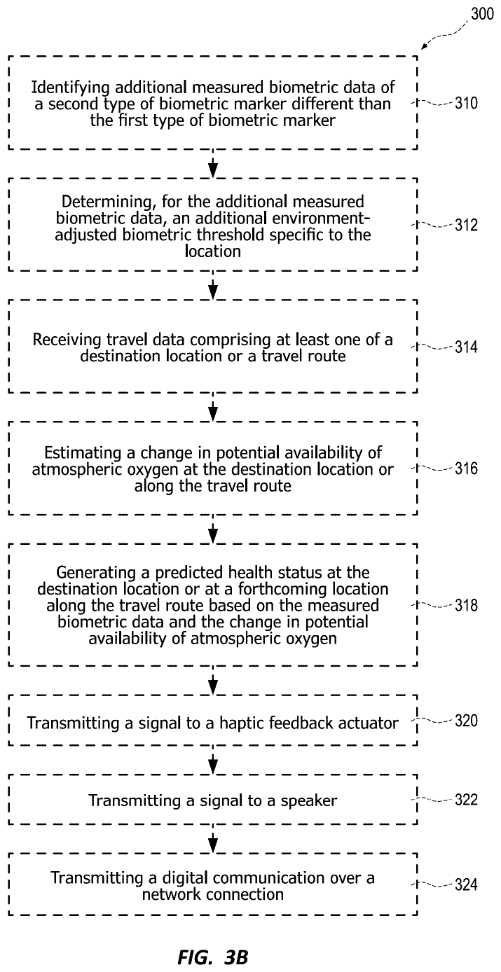

In some examples, the wearable device 102 can include a memory device 132. The memory device 132 can include one or more memory devices (e.g., individual nonvolatile memory, processor-embedded nonvolatile memory, random access memory, memory integrated circuits, DRAM chips, stacked memory modules, storage devices, memory partitions, etc.). The memory device 132 can store computer-executable instructions, including those described in this disclosure and/or those necessary to perform a particular process disclosed herein. For example, the memory device 132 can store computer-executable instructions for performing the method steps described below in relation to FIGS. 3A-3B and/or generating the example visualizations shown and described in relation to FIGS. 4-7. In at least one example, the memory device 132 can include a web application, a native application installed on the wearable device 102 (e.g., a portable device application, mobile device application, wearable device application, plug-in application, etc.), or a cloud-based application where at least part of the system functionality is performed by one or more servers.

In certain examples, the wearable device 102 can include a power supply 133. The power supply 133 can include any power source that can provide power to one or more components of the wearable device 102 (or other components of the system environment 100). For example, a power supply can include fuel cells, battery cells, generators, alternators, solar power converters, motion-based converters (e.g., that convert vibrations or oscillations into power), etc. In particular implementations, a power supply can convert alternating current to direct current (or vice-versa) for powering or charging/recharging components of the wearable device 102. Some particular examples of a power supply can include a switched mode power supply, an uninterruptible power supply, an alternating current power supply, a direct current power supply, a regulated power supply, a programmable power supply, a computer power supply, and a linear power supply. In some examples, a power supply includes a rechargeable battery (e.g., including one or more lithium-ion cells).

In these or other examples, the wearable device 102 can communicate with other devices (e.g., the computing device 106 and/or the third-party server(s) 108 shown in FIG. 1) to receive and/or transmit data. For example, and as will be discussed below, the computing device 106 can sense environmental conditions (e.g., via the environment sensor(s) 134 onboard the computing device 106) and/or retrieve environmental conditions from the third-party server(s) 108. The computing device 106 can then relay the environmental conditions to the wearable device 102 for adjusting biometric data according to the environmental conditions. Still, in other examples, the wearable device 102 can relay environment-adjusted biometric data to the computing device 106 (e.g., for display, storing, and/or software application use).

The computing device 106 can include virtually any type of computing device. In some examples, the computing device 106 can include a scent control apparatus. In other examples, the computing device 106 can include a smart phone, radio, notebook computer, desktop computer, tablet, wearable, watch, head-mountable device, audio device (e.g., ear buds, headphones, ear muffs), server, similar devices, and combinations thereof. In some examples, the computing device 106 can include a smart optics device (e.g., a gun scope, digital bow sight, binoculars, range finder, spotting scope, etc.). In at least one example, the computing device 106 can include an external sensor device, such as a chest-worn heart rate sensor, weather meter (e.g., a KESTREL® weather meter), handheld GPS unit, etc. In certain implementations, the computing device 106 can be part of a vehicle system (e.g., an airplane, helicopter, truck, ambulance, etc.).

In these or other examples, the computing device 106 can include environment sensor(s) 134, a processor 136, and/or a memory device 138. The environment sensor(s) 134 can be the same as or similar to the environment sensor(s) 116 discussed above for the wearable device 102. The processor 136 can be the same as or similar to the processor 130 discussed above for the wearable device 102. The memory device 138 can be the same as or similar to the memory device 132 discussed above for the wearable device 102.

In some examples, the wearable device 102 and/or the computing device 106 can communicate with the third-party server(s) 108. The third-party server(s) 108 can include a content server and/or a data collection server. Additionally or alternatively, the third-party server(s) 108 can include an application server, a communication server, a web-hosting server, a social networking server, or a digital content management server. In specific implementations, the third-party server(s) 108 can include a messaging server, GPS or satellite server, weather service server, RSS (really simple syndication) data feed server, etc. For example, the third-party server(s) 108 can include a cloud-based (or internet based) weather server providing real-time weather data monitoring for locations throughout the world. In these or other examples, the wearable device 102 and/or the computing device 106 can retrieve data from the third-party server(s) 108 to perform various method steps disclosed herein. Additionally or alternatively, the third-party server(s) 108 can include data centers that store historical user data and/or historical weather conditions and environment data for specific locations. In turn, the third-party server(s) 108 can provide data to the wearable device 102 and/or the computing device 106, where the provided data leverages the accuracy, repeatability, and data smoothing from many different users in a same or similar environment.

In some examples, the various elements of the system environment 100 can communicate with each other (and thereby be communicatively coupled) via the network 110. The network 110 can be any suitable network over which computing devices communicate. In these or other examples, the network 110 can include a wireless local area network, wireless area network, wireless personal area network, wide area network, etc. Some particular examples of wireless networks include a Wi-Fi based network, mesh network, BLUETOOTH® network, near-field communication network, low-energy/low power communication network, Zigbee network, Z-wave network, 6LoWPAN network, radio wave-based network, satellite network, LoRa long range communication network, etc. Other forms of the network 110 can include wired connections, such as a USB network, UART network, USART network, I2C network, SPI network, QSPI network, etc.

Any of the features, components, and/or parts, including the arrangements and configurations thereof shown in FIG. 1 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 1.

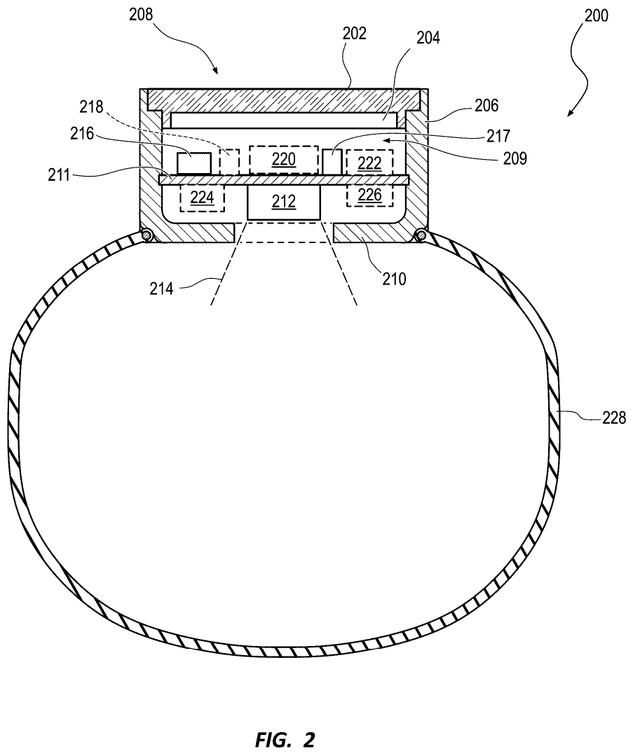

FIG. 2 illustrates a side cross-sectional view of a wearable device 200 in accordance with one or more examples of the present disclosure. The wearable device 200 is an example implementation of the wearable device 102 discussed above and has many of the same or similar elements previously discussed.

As shown, the wearable device 200 can include a cover 202. The cover 202 can include a cover glass, outer display surface, or viewing lens disposed over a display aperture 208 defined by a housing 206. In some examples, the cover 202 can include a protective element, coating, or layer positioned over a display element 204 (e.g., a light display, light emitting diode, one or more display layers, etc.). In certain examples, the cover 202 is mounted to the housing 206. In particular examples, the cover 202 is seated against the housing 206 so as to provide a seal that prevents or inhibits ingress of dirt, fluids, or other contaminants from the environment into an internal volume 209 defined by the housing 206.

In these or other examples, the housing 206 can include a frame, shell, hull, chassis, or body structure of the wearable device 200. A variety of materials for the housing 206 can be utilized, including composites, metal, plastic, and combinations thereof (for example). The housing 206 can define the metes and bounds of the internal volume 209 for at least partially enclosing and protecting the various internal components of the wearable device 200. The display aperture 208 can be defined by the upper periphery of the housing 206 opposite a bottom face 210. The bottom face 210 can be positionable against a user's body or limb (e.g., a user's wrist).

A variety of components can be disposed within the housing 206 (i.e., inside the internal volume 209). In some examples, the housing 206 can include a printed circuit board (PCB) 211 for mounting various components, providing the associated electrical circuitry to electrically couple components, and affixing such components relative to the housing 206. In certain examples, a pulse oximeter 212 (and/or other sensors) is at least partially disposed within the housing 206 and can be mounted to the PCB 211. In at least one example, the pulse oximeter 212 is communicatively coupled to a processor (e.g., the controller 216). The pulse oximeter 212 can include a field of view 214 (e.g., a range, depth, and/or coverage of sensor signals) that can extend through the bottom face 210 of the housing 206. That is, in some examples, the bottom face 210 can define an opening sized and shaped for the pulse oximeter 212 such that sensor signals (e.g., optical signals) can pass through the opening in the bottom face 210. In other examples, the bottom face 210 does not include an opening, but can be optically transparent to allow optical signals to be transmitted and/or received by the pulse oximeter 212 through the material of the bottom face 210.

A controller 216 can be disposed within the housing 206. The controller 216 can include a processor and memory device (e.g., the processor 130 and the memory device 132 discussed above) for controlling operation of the wearable device 200. In particular examples, the controller 216 can control various components of the wearable device 200 and communicate signals therebetween. In at least one example, the controller 216 includes computer-executable instructions for receiving optical data from the pulse oximeter 212, identifying an altitude-adjusted oxygen saturation factor specific to an altitude of the wearable device 200, determining an altitude-adjusted oxygen saturation level based on the optical data and the altitude-adjusted oxygen saturation factor, and providing (at the display element 204) a graphical user interface that includes an oxygen indicator representing the altitude-adjusted oxygen saturation level. These and/or other examples are discussed further below in relation to subsequent figures.

In some examples, the wearable device 200 can include a power supply 217 (the same as or similar to the power supply 133 discussed above). The power supply 217 can provide power to the various components of the wearable device 200.

In some examples, the wearable device 200 can additionally include a communications element 218. The communications element 218 can include an antenna, resonating element, transponder, transceiver, transmitter/receiver, network connection components (e.g., cellular modem), SIM card (or virtual/embedded eSIM card), etc. The communications element 218 can allow communications between the wearable device 200 and external devices over a network connection (e.g., the network 110 discussed above).

In at least one example, the wearable device 200 can include an environment sensor 220, speaker 222, microphone 224, and a haptic feedback actuator 226 (all disposed within the internal volume 209, and in some cases mounted to the PCB 211 and/or housing 206). The environment sensor 220, speaker 222, microphone 224, and haptic feedback actuator 226 can respectively be the same as or similar to the environment sensor(s) 116, the speaker 126, the microphone 128, and the haptic feedback actuator 124 discussed above in relation to FIG. 1.

Further shown, the wearable device 200 can include a band 228. The band 228 can secure the wearable device 200 to a user (e.g., the user's wrist, torso, etc.). In some examples, the band 228 is removable, interchangeable, customizable, etc.

Any of the features, components, and/or parts, including the arrangements and configurations thereof shown in FIG. 2 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 2.

FIGS. 3A-3B, the corresponding text, and the examples provide several different systems, methods, techniques, components, and/or devices of an environment-specific biometric detection system in accordance with one or more embodiments of the present disclosure. In addition to the above description, one or more embodiments can also be described in terms of flowcharts including acts for accomplishing a particular result or performing a certain function. For example, FIGS. 3A-3B illustrate a flowchart of a series of acts 300 for performing environment-specific biometric detection in accordance with one or more embodiments of the present disclosure. One or more examples of a wearable device and/or a computing device (e.g., the wearable device 102, the computing device 106, the wearable device 200, etc.) may perform one or more acts of the series of acts 300 in addition to or alternatively to one or more acts described in conjunction with other figures. In other examples, one or more servers, remote data centers, third-party servers, etc. may perform one or more acts of the series of acts 300 alternatively to, or in addition to (e.g., in parallel with or in series with), the series of acts 300 performed by one of a wearable device and/or a computing device disclosed herein. While FIGS. 3A-3B illustrates acts according to one embodiment, alternative embodiments may omit, add to, reorder, and/or modify any of the acts shown in FIGS. 3A-3B. The acts of FIGS. 3A-3B can be performed as part of a method. Alternatively, a non-transitory computer-readable medium can include instructions that, when executed by one or more processors, cause a computing device (or a computer component, such as a processor, implemented on a wearable device) to perform the acts of FIGS. 3A-3B. In some embodiments, a system can perform the acts of FIGS. 3A-3B.

As shown in FIG. 3A, the series of acts 300 can include an act 302 of determining a real-time (e.g., current or near-current) availability of atmospheric oxygen for a location based on environmental conditions (e.g., altitude, oxygen concentration, humidity, barometric pressure, etc.). In these or other examples, a location can be a precise location (e.g., GPS coordinate) or an approximated location (e.g., within a threshold distance, elevation, etc.) that can be associated with a user's location. The level of approximation or proximity to a user location can depend on terrain, cloud or atmospheric conditions, obstacles, network connection or signal strength, etc. In certain implementations, a location can be input by a user (e.g., an address, mile marker, GPS coordinate, trail, or other location self-approximated within a certain proximity).

The act 302 can be achieved in a variety of ways. In some examples, determining the real-time availability of atmospheric oxygen is performed by using on-board sensors to directly measure a local oxygen concentration (e.g., using an oxygen sensor, gas sensor, etc.). In other examples, determining the real-time availability of atmospheric oxygen is performed by using on-board sensors (e.g., altimeter, pressure sensor, etc.) to indirectly measure an availability of oxygen in the atmosphere. For instance, using one or more onboard sensors (i.e., onboard the wearable device and/or onboard a computing device communicatively coupled to the wearable device), at least one of an altitude, humidity, temperature, barometric pressure, or other environmental condition can be measured. From the measured environmental condition(s), a corresponding availability of atmospheric oxygen can be accurately identified. To illustrate, onboard sensors can identify one or more terms of the Ideal Gas Law equation (e.g., pressure and temperature for a known sensing volume) to solve for the amount of atmospheric oxygen content (e.g., the amount of oxygen molecules). The determined amount of oxygen molecules can then be adjusted to account for the environment's real-time relative humidity levels because humidity displaces oxygen molecules. In this way, a highly accurate calculation of atmospheric oxygen content can be obtained.

In some examples, onboard sensors can determine a barometric pressure (term P in the expression below) and humidity level (term PH2O representing water vapor pressure in the expression below). A processor can then use the following expression (stored in a memory device) to identify the partial pressure of atmospheric oxygen (term PO2 representing oxygen concentration): PO2=(P−PH2O)*0.2095. In certain implementations, indirectly measuring the availability of oxygen in the atmosphere in this way using other environmental conditions is more accurate than oxygen/gas sensors that can directly measure the availability of atmospheric oxygen.

In at least some cases, determining the environmental conditions at the act 302 can include retrieving data from one or more third-party servers (e.g., the third-party server(s) 108 discussed above). Specifically, the act 302 can include retrieving, for a real-time location of a computing device (e.g., the computing device 106 discussed above), one or more environmental conditions via a network connection to a network device-such as a GPS server, weather server, etc. As an example, the act 302 can include identifying global positioning system (GPS) coordinates retrieved from a GPS server or GPS satellite device. In turn, the computing device (or wearable device) can ping a weather server to retrieve real-time weather conditions (e.g., pressure, humidity, etc.) for the exact GPS coordinates. Alternatively, the weather server ping can be omitted. Instead, GPS coordinates can be used to determine the location, the altitude above sea level can be determined based on the location, and the real-time availability of oxygen as an amount of oxygen concentration at the altitude above sea level can be determined. For instance, using the GPS coordinates alone (or instead location data input from the user), the computing device can utilize installed map data—including topographical map data or altitude data—to cross-reference the retrieved GPS coordinates with altitude data in the map data. In turn, the computing device can relate the altitude (above sea level) data to an amount of oxygen concentration at the altitude above sea level using one or more solvers, data tables, etc.

The series of acts 300 can include an act 304 of identifying measured biometric data of a user (e.g., the user while currently positioned at the location or within a threshold proximity of the location). In some examples, the act 304 can include the wearable device using one or more sensors to detect biometric signals. For instance, the act 304 can include the wearable device using an oximeter to determine an oxygen saturation (SPO2) value for the user. Additionally or alternatively, the act 304 can include the wearable device using photoplethysmography sensors to estimate a heart rate of the user. Many other sensors and biometric markers for a user can be identified. In some examples, biometric data can be retrieved from other devices (e.g., the computing device 106).

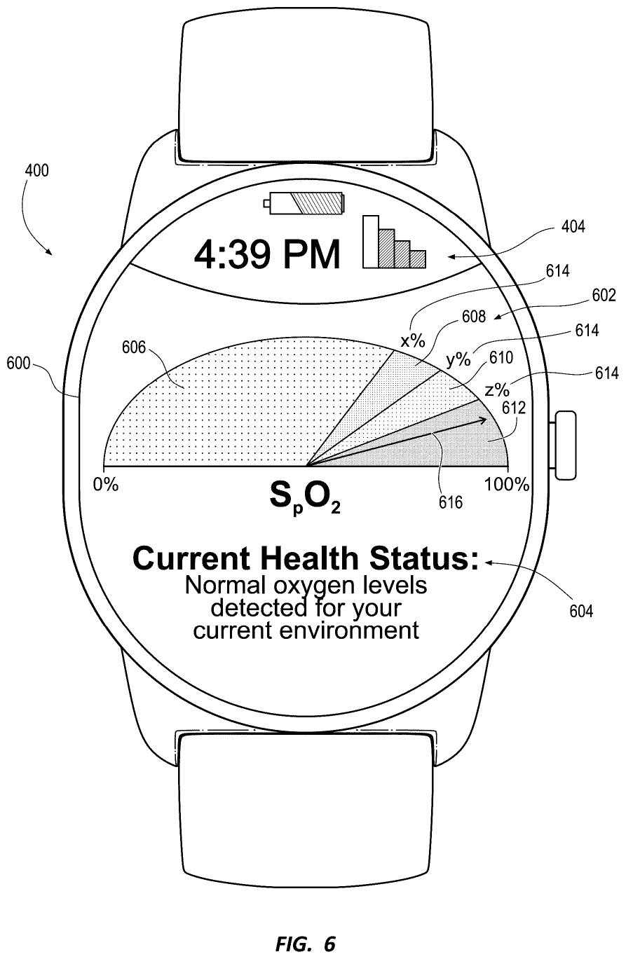

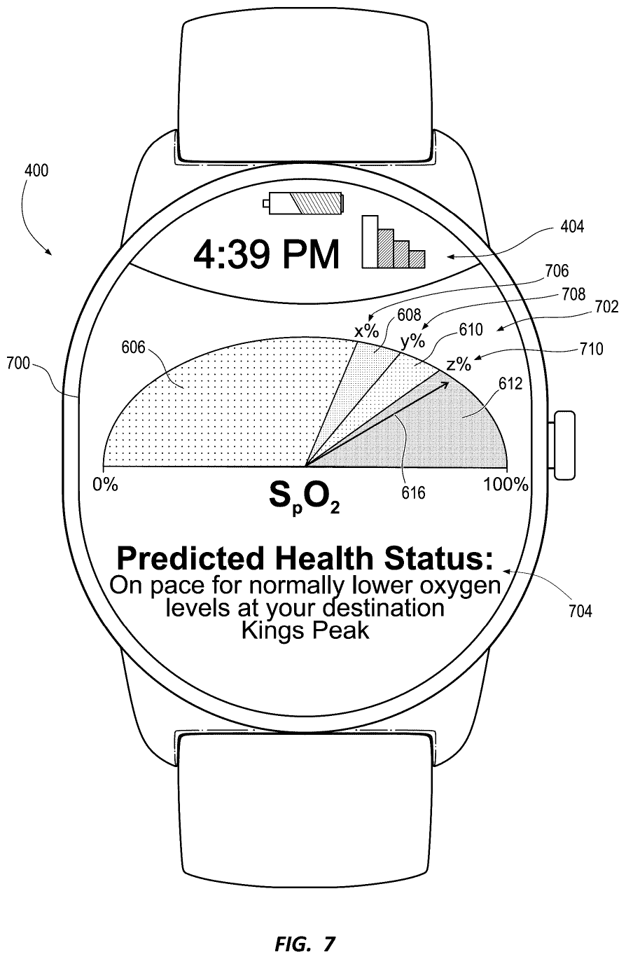

The series of acts 300 can include an act 306 of determining an environment-adjusted biometric threshold specific to the location. An environment-adjusted biometric threshold can refer to a parameter, range, category, limit, or numerical bound associated with biometric data that is modified according to the real-time environmental conditions. For example, an environment-adjusted biometric threshold can refer to modified ranges of biometric values (e.g., as seen in FIGS. 6-7) that may differ based on the location and corresponding environmental conditions. Environment-adjusted biometric thresholds—which account for the user environment—can thus differ from ranges of biometric markers that are considered “normal,” “textbook,” or “typical” because these values inherently are universally agnostic to user environments.

Specific implementations of environment-adjusted biometric thresholds can include modified biometric ranges that indicate a user status (e.g., a healthy status, normal status, emergency status, medical attention needed status, etc.)—where the user status based on a measured biometric value can vary depending on the location or environment of the user. In other terms (and using a specific example), a particular oxygen saturation level of 93% might be considered slightly below normal in San Diego, California (at sea level), but perfectly acceptable in the Rocky Mountains (e.g., at 10,000 feet above sea level). Accordingly, the act 306 can include the various steps to determine precisely how to calibrate biometric thresholds to account for the environment of the user—thereby facilitating more accurate information to provide to the user by qualifying the biometric data that is shown.

Additionally or alternatively to adjusting the biometric thresholds for biometric data, the act 306 can include adjusting the actual biometric data itself for the environment. An example of environment-adjusted biometric data can include environment-adjusted vital signs. Using the above example, rather than display a measured value of 93% oxygen saturation while located in the Rocky Mountains, the wearable device may determine an environment-adjusted oxygen saturation level of 98% to display that accounts for the increased elevation (and therefore the decreased amount of available atmospheric oxygen content to the user). This adjustment of the biometric data can more accurately reflect how the user is performing or adapting to a specific environment. Indeed, without such environment calibration of a vital sign, a user would be unable to ascertain what aspects of a specific biometric marker can be attributed to limitations imposed by a user's environment (as opposed to their body's own unique limitations). In the specific example above, the Rocky Mountains environment imposed a 5% differential in oxygen saturation level that the user would wrongly attribute to limitations of their body. Thus, environment-adjusted biometric data can more accurately reflect the true performance limitations of a user given specific environmental conditions.

The act 306 can be accomplished in various ways. In some examples, at least part of the act 306 is performed on a device external to the wearable device (e.g., on the computing device 106 communicatively coupled to the wearable device 102). In one such example, the act 306 includes determining an environment-adjusted vital sign factor specific to the location and transmitting the environment-adjusted vital sign factor to the wearable device. In other examples, the act 306 is performed entirely on the wearable device (e.g., utilizing one or more solvers, native-installed applications, etc.).

The term “environment-adjusted vital sign factor” can refer to an environment-specific conversion factor that can be used to modify vital sign thresholds and/or the vital sign data itself (as discussed above). In some examples, the environment-adjusted vital sign factor is based on programmed data tables (e.g., utilizing the ideal gas law equation for pressure versus altitude). For instance, the environment-adjusted vital sign factor can be a ratio of effective oxygen percentage at altitude relative to the effective oxygen percentage at sea level (as shown in the following TABLE 1):

TABLE 1

| | | Environment- |
|---|---|---|
| | | Adjusted |
| Altitude | Effective | Vital Sign |
| (feet) | Oxygen % | Factor |
| 0 | 20.9 | 1 |
| 1000 | 20.1 | 0.9617 |
| 2000 | 19.4 | 0.9282 |
| 3000 | 18.6 | 0.8900 |
| 4000 | 17.9 | 0.8565 |
| 5000 | 17.3 | 0.8278 |
| 6000 | 16.6 | 0.7943 |
| 7000 | 16 | 0.7656 |
| 8000 | 15.4 | 0.7368 |
| 9000 | 14.8 | 0.7081 |
| 10000 | 14.3 | 0.6842 |
| 11000 | 13.7 | 0.6555 |
| 12000 | 13.2 | 0.6316 |
| 13000 | 12.7 | 0.6077 |
| 14000 | 12.3 | 0.5885 |
| 15000 | 11.8 | 0.5646 |
| 16000 | 11.4 | 0.5455 |
| 17000 | 11 | 0.5263 |
| 18000 | 10.5 | 0.5024 |
| 19000 | 10.1 | 0.4833 |
| 20000 | 9.7 | 0.4641 |
| 21000 | 9.4 | 0.4498 |
| 22000 | 9 | 0.4306 |
| 23000 | 8.7 | 0.4163 |
| 24000 | 8.4 | 0.4019 |
| 25000 | 8.1 | 0.3876 |
| 26000 | 7.8 | 0.3732 |
| 27000 | 7.5 | 0.3589 |
| 28000 | 7.2 | 0.3445 |
| 29000 | 6.9 | 0.3301 |

The wearable device and/or the computing device can then use the environment-adjusted vital sign factor to adjust or modify the biometric data itself (and/or to generate environment-adjusted biometric thresholds). For instance, the environment-adjusted vital sign factor can be a multiplier that, when applied to the biometric data itself (and/or certain biometric thresholds), modifies the biometric data and/or biometric thresholds to reflect the specific environmental conditions of the user. As a specific example, the wearable device can apply the environment-adjusted vital sign factor (in this case, an altitude-adjusted oxygen saturation factor) to SpO2 values determined via optical data from a pulse oximeter to determine an altitude-adjusted oxygen saturation level.

Environment-adjusted vital sign factors are not limited to the above example. Other examples of an environment-adjusted vital sign factors can include one or more biometric markers that reflect the current conditions of the user's environment (and can therefore be used to convert to other environment-adjusted values of interest). PaCO2, known as the partial pressure of carbon dioxide, is one example of a biometric marker that can be used to generate, for example, an altitude adjusted SpO2 value. Specifically, PaCO2 can be a biometric marker that is detected by the wearable device utilizing a transcutaneous CO2 sensor (PtCO2), such as a Severinghause electrode in the wearable device. In turn, SpO2 values can then be determined (e.g., using the Severinghaus equation) from the following estimation of PaO2, which is the partial pressure of oxygen in arterial blood:

$$PaO 2 = PaO 2 - ( A - a Gradient ) ,$$

where the term PAO2 represents the alveolar partial pressure of oxygen, and the term (A−a Gradient) is an age-based constant (i.e., [Age/4]+4). PAO2 can be estimated as follows:

$$PaO 2 = FiO 2 ( Patm - PH 2 O ) - ( PaCO 2 / 0.8 ) ,$$

where the term FiO2 represents the effective oxygen percentage in Table 1 above for a given atmospheric pressure (Patm), the term PH2O represents the environment's water vapor pressure, and the term PaCO2 represents the partial pressure of carbon dioxide. PaCO2 can be measured via an arterial blood gas test, but PaCO2 can also be estimated using a capnograph to measure end-tidal CO2 (EtCO2), utilizing a transcutaneous CO2 sensor (PtCO2) such as a Severinghause electrode in the wearable device, or utilizing the user's bicarbonate level (HCO3-) in combination with formulas like Winter's formula.

The series of acts 300 can include an act 308 of generating, for display in a graphical user interface of a wearable device, a visualization (e.g., the visualization 122) based on the measured biometric data and the environment-adjusted biometric threshold. In one or more examples, the act 308 can include comparing the measured biometric data to the environment-adjusted biometric threshold. In certain examples, the wearable device can generate a first visualization in the event that the measured biometric data fails to satisfy an environment-adjusted biometric threshold, and a second (different) visualization in the event that the measured biometric data satisfies an environment-adjusted biometric threshold.

In some examples, the visualization includes a recommendation regarding user exertion. The exertion-related recommendation may be to push harder, run faster, climb higher, etc. In other examples, the recommendation may be to slow or ease exertion (e.g., as shown in FIG. 4). In some examples, the visualization can include a recommendation to increase oxygen intake (e.g., as also shown in FIG. 4), seek lower elevation, take deeper breaths, follow a certain trail, etc. In at least one example, the visualization includes an indicator graphically representing the biometric data in relation to the environment-adjusted biometric threshold (e.g., as shown in FIGS. 6-7). In one example, the indicator can include an altitude-adjusted vital sign (e.g., an altitude adjusted SpO2 value). In specific implementations, the visualization further includes an altitude indicator for the location.

In certain examples, the act 308 can additionally (or alternatively) include the wearable device transmitting environment-adjusted vital sign data of the user—where the environment-adjusted vital sign data can include vital sign data of the user that is modified according to the environment-adjusted vital sign factor discussed above—to a computing device (e.g., the computing device 106). In such examples, the computing device can log or track the environment-adjusted vital sign data of the user over time, create training modules, display progress reports/charts, relay to third-party data servers, etc. Additionally or alternatively, the computing device may generate one or more of the same or similar visualizations discussed above via a corresponding application installed on the computing device.

In certain examples of the act 308, the computing device (and/or a server disclosed herein) can collect the environment-adjusted vital sign data, corresponding environmental conditions of the user, or any other user-environment related data. The collected data can enhance future predictions for the user and/or for other users (e.g., as more data points are collected). In certain examples, for instance, the collected data can be used to train machine-learning models, optimization models, etc. that can enhance predictive attributes of one or more models, solvers, etc. disclosed herein. In at least one example, the collected data can be used to learn more about a given user and improve associated predictions for that user (e.g., by “learning” how the user adapts to or performs in various environments over time).

FIG. 3B shows example optional acts in the series of acts 300. Some, all, or none of the acts shown in FIG. 3B may be performed. According to some examples, the series of acts 300 can include an act 310 of identifying additional measured biometric data of a second type of biometric marker different than the first type of biometric marker. For example, the first type of biometric marker and the second type of biometric marker can be any combination of any of the biometric markers disclosed herein. Many different biometric markers can be used in combination with each other. In one such example, the first type of biometric marker can be oxygen saturation and the second type of biometric marker can be heart rate.

The series of acts 300 can include an act 312 of determining, for the additional measured biometric data, an additional environment-adjusted biometric threshold specific to the location. In a same or similar fashion to the act 306 discussed above, the wearable device and/or the computing device can determine an additional environment-adjusted biometric threshold. For instance, a user's heart rate can increase at higher elevations to compensate for the lower availability of atmospheric oxygen (i.e., by delivering more blood flow to deliver the same amount of oxygen as at lower elevations). Thus, adjusted heart rate thresholds can be modified to account for elevated heart rates at altitude (e.g., so that a user can accurately understand how their heart is performing in any specific environment).

The series of acts 300 can include an act 314 of receiving travel data including at least one of a destination location or a travel route. In some examples, the act 314 can include receiving user input of the destination location or the travel route. In other examples, the act 314 can include inferring the destination location or the travel route based on a current location, compass heading or direction, speed of travel, elevation climb/decline, available trails or roads within a threshold proximity, etc.

The series of acts 300 can include an act 316 of estimating a change in potential availability of atmospheric oxygen at the destination location or along the travel route. In some examples, the act 316 can include estimating a time of arrival to the destination location or a segment of interest along the travel route (where the estimated time of arrival may be based on a current location, compass heading or direction, speed of travel, elevation climb/decline, available trails or roads within a threshold proximity, etc.). In some examples, the estimated time of arrival can be determined using an application programming interface that connects the wearable device and/or the computing device to a map data server (e.g., GOOGLE MAPS® server) for providing the estimated time of arrival. In turn, the act 316 can include obtaining forecasted weather conditions corresponding to the estimated time of arrival (e.g., pressure, temperature, humidity, etc.)—in addition to or alternatively to an altitude of the destination location. Then, in a same or similar manner as discussed above for the act 302, the wearable device and/or the computing device can determine the real-time availability of atmospheric oxygen for the destination location or a segment of interest along the travel route.

The series of acts 300 can include an act 318 of generating a predicted health status at the destination location or at a forthcoming location along the travel route based on the measured biometric data and the change in potential availability of atmospheric oxygen. In some examples, the act 318 can include estimating biometric data at the destination location (e.g., based on currently measured and/or historically measured biometric data). For instance, the act 318 can include extrapolating the biometric data as currently trending to a future point in time corresponding to the estimated time of arrival at the destination location or the segment of interest along the travel route. In turn, the act 318 can include comparing the estimated biometric data against environment-adjusted biometric thresholds (which are again modified according to the destination location or segment of interest along the travel route). Based on the comparison, the act 318 can include generating a predicted health status. The predicted health status can coincide with how the wearable device and/or the computing device predicts the user's future biometric data will relate to environment-adjusted biometric thresholds that are specific to the destination location (or segment of interest along the travel route).

The series of acts 300 can include an act 320 of transmitting a signal to a haptic feedback actuator. For example, in response to determining that an altitude-adjusted oxygen saturation level satisfies a predetermined altitude-adjusted oxygen saturation level, the wearable device can transmit a signal to actuate the haptic feedback actuator (e.g., causing a perceived vibration for the user).

The series of acts 300 can include an act 322 of transmitting a signal to a speaker. For example, in response to determining that an altitude-adjusted oxygen saturation level satisfies a predetermined altitude-adjusted oxygen saturation level, the wearable device can transmit a signal to a speaker causing a perceived audible communication (e.g., a noise, sound, notification, warning, etc.) for the user.

The series of acts 300 can include an act 324 of transmitting a digital communication over a network connection. For example, in response to determining that an altitude-adjusted oxygen saturation level satisfies a predetermined altitude-adjusted oxygen saturation level, the wearable device can transmit a digital communication (e.g., a text message, voice call, video call, sound recording, automated message, emergency message, transponder signal, etc.) over the cellular network connection or the satellite network connection in response to determining the altitude-adjusted oxygen saturation level satisfies a predetermined altitude-adjusted oxygen saturation level.

As mentioned above, the series of acts 300 can be modified in many different ways. In some examples, no biometric data of the user is required. Environment-adjusted biometric data can be generated solely on the environment, in accordance with one or more examples of the present disclosure. For instance, oxygen saturation at any given altitude can be estimated (without any measured biometric data) using the following expression:

$$SPO 2 = 100 * ( \frac{0.21 * ( 760 - 0.5 * Altitude )}{1 + 0.004 * ( 37 - Temperature )} )$$

Another example expression can also be utilized to estimate oxygen saturation at a given altitude without any biometric data. The following example expression is an estimation of arterial oxygen saturation in relation to altitude, where arterial oxygen saturation levels (typically measured using an arterial blood gas test) can be converted to SpO2.

$$Approx Arterial Blood Oxygen Saturation = 103.3 - ( altitude \times 0.0047 ) + ( Z ) ,$$

where the term (Z) is a constant value of 0.7 for men and 1.4 for women. Still, other example expressions can be utilized for estimating oxygen saturation at a given altitude without any biometric data.

As discussed above, a wearable device can perform certain operations for accomplishing a particular result or performing a certain function (e.g., performing environment-specific biometric detection). In some examples, a wearable device of the present disclosure can include a client application that includes computer-executable instructions that (upon execution) cause the wearable device to present a graphical user interface of the client application. The following description of FIGS. 4-7 is illustrative.

FIG. 4 illustrates an example graphical user interface 402 of a wearable device 400 in accordance with one or more examples of the present disclosure. As shown the graphical user interface 402 can include device information 404 (e.g., battery status, time, date, cellular connection, etc.). In certain examples, the device information 404 can include other/external device information (e.g., of a computing device, scent control apparatus, rifle scope, binoculars, or other device communicatively coupled to the wearable device 400).

In some examples, the graphical user interface 402 can include a visualization 406. The visualization 406 can include a user recommendation (in this case, “Exertion too high. Take a rest or increase oxygen intake.”). In these or other examples, the wearable device 400 can generate the visualization 406 based on environment-adjusted vital sign data (as discussed above). In specific implementations, the visualization 406 can be based on a travel route to a destination location (therefore accounting for current and future availability of atmospheric oxygen as discussed above in relation to acts 314-318 of FIG. 3B. Those of ordinary skill in the art having the benefit of this disclosure will recognize that many other visualizations are herein contemplated.

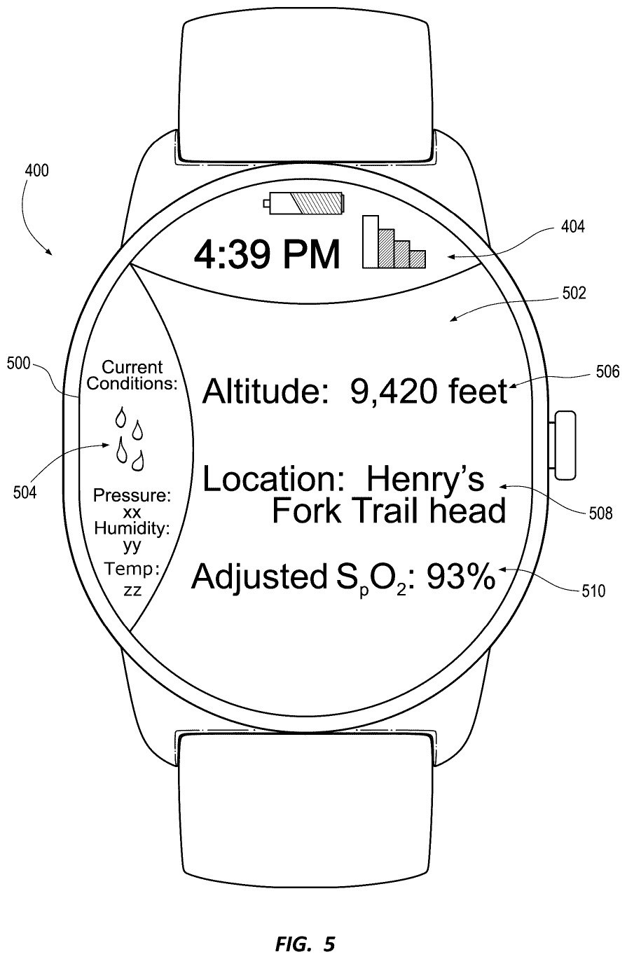

FIG. 5 illustrates an example graphical user interface 500 of the wearable device 400 in accordance with one or more examples of the present disclosure. As shown, the graphical user interface 500 can include a visualization 502. The visualization 502 can include local conditions 504 (e.g., local environmental conditions such as rain, pressure, humidity, temperature, etc. for a current location 508). The visualization 502 can include an altitude indicator 506 (e.g., a numerical indicator or graphical icon indicative of the local altitude for the location 508). Those of ordinary skill in the art having the benefit of this disclosure will recognize that the current location 508 can be represented in many different ways. In some examples, the current location 508 can be a trail, a point along the trail (e.g., the trailhead), a road, neighborhood, borough, city, mountain range, coastline section, river, or other geographical or location marker. In certain examples, the current location 508 can be GPS coordinates (e.g., latitude and longitude coordinates), geodetic coordinates, transverse Mercator coordinates, Lambert conformal conic coordinates, or other coordinate-type information, such as degrees, minutes, and seconds.

In one or more examples, the visualization 502 can further include an altitude-adjusted oxygen indicator 510. The altitude-adjusted oxygen indicator 510 can, as shown, include an oxygen saturation level that is specific to the location 508 where the elevation is 9,420 feet above sea level. In this case, the altitude-adjusted oxygen indicator 510 is 93% (e.g., 93% of a modified 100% threshold because at this altitude of 9,420, the 100% level possible for oxygen saturation is less than at sea level). Because of the altitude-adjusted oxygen indicator 510, a user can more readily (and more accurately) ascertain how their body is performing in a specific environment.

FIG. 6 illustrates yet another example graphical user interface 600 of a wearable device in accordance with one or more examples of the present disclosure. As shown, the graphical user interface 600 can include a visualization 602 that includes a current health status 604. The current health status 604 can be based on a comparison of measured biometric data (represented by measured biometric indicator 616) to one or more environment-adjusted biometric thresholds (e.g., environment-adjusted biometric thresholds 614). In some examples, the environment-adjusted biometric thresholds 614 can include at least one of a threshold SpO2 value or a range of SpO2 values indicative of a user status that is specific to the location. In certain examples, the comparison may identify that the measured biometric data indicates that the user falls in one or more user status categories 606-612. In some examples, the user status includes at least one of normal oxygen levels detected (e.g., user status category 612), low oxygen levels detected (e.g., user status category 610), hypoxemia detected (e.g., user status category 608), or medical attention required (e.g., user status category 606).

FIG. 7 illustrates still another example graphical user interface 700 of a wearable device in accordance with one or more examples of the present disclosure. As shown, the graphical user interface 700 can include a visualization 702 that includes a predicted health status 704. The predicted health status 704 can be generated in a same or similar manner as discussed above in relation to acts 314-318 of FIG. 3B. In a particular example, the wearable device can update the environment-adjusted biometric thresholds 614 (which were specific to a particular current location) to reflect a destination location (e.g., “Kings Peak”). These updated environment-adjusted biometric thresholds are represented by environment adjusted biometric thresholds 706-710 corresponding to the user status categories 606-612. In the visualization 702, however, the environment adjusted biometric thresholds 706-710 have been shifted to the left (i.e., counterclockwise) due to the increased elevation (and therefore decreased availability of atmospheric oxygen) at the destination location Kings Peak. Thus, although the measured biometric indicator 616 is predicted to fall (i.e., shift left or counter-clockwise), the corresponding user status category is predicted to be the user status category 612 at the destination location which may represent normally lower oxygen levels given the availability of atmospheric oxygen predicted at the destination location.

In some examples, a user can more accurately and readily plan for arrival at the destination location based on the predicted health status 704. The user can identify if their body is on pace to acclimate to their destination location or if other travel plans should be arranged. Accordingly, a wearable device (and/or computing device) as disclosed herein—having the ability to provide environment-adjusted biometric thresholds and/or environment-adjusted biometric data—can provide more accurate, relevant biometric-related information to a user for a wide variety of use cases and applications than heretofore achieved.

Any of the features, components, and/or parts, including the arrangements and configurations thereof shown in FIGS. 4-7 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 4-7.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not intended to be exhaustive or to limit the embodiments to the precise forms disclosed.

It will be apparent to one of ordinary skill in the art that many modifications and variations are possible in view of the above teachings. Indeed, various inventions have been described herein with reference to certain specific aspects and examples. However, they will be recognized by those skilled in the art that many variations are possible without departing from the scope and spirit of the inventions disclosed herein. Specifically, those inventions set forth in the claims below are intended to cover all variations and modifications of the inventions disclosed without departing from the spirit of the inventions. The terms “including” or “includes” as used in the specification shall have the same meaning as the term “comprising.” Additionally, the terms “about,” “approximately,” and “substantially” should be interpreted as +/−10 percent of a given value, unless otherwise indicated.

What is claimed is:

1. A method, comprising:
determining a real-time availability of atmospheric oxygen for a location based on environmental conditions;
identifying measured biometric data of a user currently positioned at the location or within a threshold proximity of the location;
determining an environment-adjusted biometric threshold specific to the location; and
generating, for display in a graphical user interface of a wearable device, a visualization based on the measured biometric data and the environment-adjusted biometric threshold.

2. The method of claim 1, wherein the visualization comprises a recommendation regarding user exertion.

3. The method of claim 1, wherein the visualization comprises a recommendation to increase oxygen intake.

4. The method of claim 1, wherein the visualization comprises an indicator graphically representing the biometric data in relation to the environment-adjusted biometric threshold.

5. The method of claim 4, wherein the indicator comprises an altitude-adjusted vital sign, and the visualization further comprises an altitude indicator for the location.

6. The method of claim 1, wherein the measured biometric data comprises an oxygen saturation (SpO2) value and the environment-adjusted biometric threshold comprises at least one of a threshold SpO2 value or a range of SpO2 values indicative of a user status specific to the location.

7. The method of claim 6, wherein the user status comprises at least one of normal oxygen levels detected, low oxygen levels detected, hypoxemia detected, or medical attention required.

8. The method of claim 1, wherein the measured biometric data is a first type of biometric marker, and the method further comprises:
identifying additional measured biometric data of a second type of biometric marker different than the first type of biometric marker; and
determining, for the additional measured biometric data, an additional environment-adjusted biometric threshold specific to the location.

9. The method of claim 8, wherein the first type of biometric marker is oxygen saturation and the second type of biometric marker is heart rate.

10. The method of claim 1, further comprising:
receiving travel data comprising at least one of a destination location or a travel route;
estimating a change in potential availability of atmospheric oxygen at the destination location or along the travel route; and
generating a predicted health status at the destination location or at a forthcoming location along the travel route based on the measured biometric data and the change in potential availability of atmospheric oxygen.

11. The method of claim 1, wherein the environmental conditions comprise altitude above sea level, and the method further comprises:
using global positioning system (GPS) coordinates to determine the location;
identifying the altitude above sea level based on the location; and
determining the real-time availability of oxygen as an amount of oxygen concentration at the altitude above sea level.

12. The method of claim 1, wherein the environmental conditions comprise at least one of altitude, oxygen concentration, or barometric pressure; and
the method further comprises using a sensor to analyze the environmental conditions, the sensor comprising at least one of an oxygen sensor, an altimeter, or a pressure sensor.

13. A system, comprising:
a wearable device; and
a computing device communicatively coupled to the wearable device, the computing device comprising:
a processor; and
a memory device storing computer-executable instructions that, when executed by the processor, cause the processor to:
retrieve, for a real-time location of the computing device, environmental conditions via a network connection to a network device;
determine a real-time availability of atmospheric oxygen for the real-time location based on the environmental conditions;
determine an environment-adjusted vital sign factor specific to the location; and
transmit the environment-adjusted vital sign factor to the wearable device.

14. The system of claim 13, further storing computer-executable instructions that, when executed by the processor, cause the processor to:
receive, from the wearable device, environment-adjusted vital sign data of a user, the environment-adjusted vital sign data comprising vital sign data of the user that is modified according to the environment-adjusted vital sign factor.

15. The system of claim 14, further storing computer-executable instructions that, when executed by the processor, cause the processor to generate a user recommendation based on the environment-adjusted vital sign data.

16. The system of claim 15, wherein the user recommendation is based on a travel route to a destination location.

17. A wearable device, comprising:
a housing defining a display aperture and an interior volume, the housing including a bottom-face;
a display positioned within the display aperture;
a pulse oximeter positioned at least partially within the interior volume and oriented opposite the display aperture to send and receive optical signals through the bottom face;
a processor disposed within the interior volume and communicatively coupled to the pulse oximeter; and
a memory device storing computer-executable instructions that, when executed by the processor, cause the processor to:
receive optical data from the pulse oximeter;
identify an altitude-adjusted oxygen saturation factor specific to an altitude of the wearable device;
determine an altitude-adjusted oxygen saturation level based on the optical data and the altitude-adjusted oxygen saturation factor; and
provide, at the display, a graphical user interface comprising an oxygen indicator representing the altitude-adjusted oxygen saturation level.

18. The wearable device of claim 17, wherein the wearable device comprises a haptic feedback actuator, and the wearable device stores computer-executable instructions that, when executed by the processor, cause the processor to transmit a signal to the haptic feedback actuator in response to determining the altitude-adjusted oxygen saturation level satisfies a predetermined altitude-adjusted oxygen saturation level, the signal configured to actuate the haptic feedback actuator.

19. The wearable device of claim 17, wherein the wearable device comprises a speaker, and the wearable device stores computer-executable instructions that, when executed by the processor, cause the processor to transmit a signal to the speaker in response to determining the altitude-adjusted oxygen saturation level satisfies a predetermined altitude-adjusted oxygen saturation level, the signal configured to induce an audible communication from the speaker.

20. The wearable device of claim 17, wherein the wearable device comprises at least one of a cellular network connection or a satellite network connection, and the wearable device stores computer-executable instructions that, when executed by the processor, cause the processor to transmit a digital communication over the cellular network connection or the satellite network connection in response to determining the altitude-adjusted oxygen saturation level satisfies a predetermined altitude-adjusted oxygen saturation level.
